(19) United States
(12) Patent Application Publication
(10) Pub. No.: US 2006/0068014 A1
(43) Pub. Date: Mar. 30, 2006

(54) Ionic hydrogels with controlled aqueous fluid absorption

(75) Inventors: Hugh Semple Munro, Chipping Campden (GB); Richard Hoskins, Wiltshire (GB)

Correspondence Address: BROWDY AND NEIMARK, P.L.L.C.;624 NINTH STREET, NW, SUITE 300, WASHINGTON, DC, 20001-5303, US

(73) Assignee: FIRST WATER LIMITED, WILTSHIRE (GB)

(21) Appl. No.: 10/515,028

(22) PCT Filed: May 20, 2003
(86) PCT No.: PCT/GB03/02218

§ 371 Date: Aug. 22, 2005

(30) Foreign Application Priority Data
May 20, 2002 (GB) 0211529.3

(51) Int. Cl. C07C 69/52; A61K 9/14

(52) U.S. Cl. 424/487; 560/222

(57) ABSTRACT

A cross-linked plasticised polymeric hydrogel suitable for use in mammalian body tissue (e.g. skin) contacting applications, which comprises a cross-linked copolymer formed from a first monomer comprising one or more pendant anionic group and a second monomer comprising one or more pendant cationic group, the relative amounts of the said monomers in the copolymer being such that the anionic groups and cationic groups are present in essentially equimolar quantities.

### FIELD OF THE INVENTION

The present invention relates to polymeric hydrogels, and more particularly to cross-linked polymeric hydrogels, for contacting mammalian body tissue, e.g. skin or flesh. Such hydrogels may, for example, be used in association with medical, health and personal care products such as patches for cosmetic devices, sensing electrodes, stimulation electrodes, devices for iontophoretic delivery of active agents, passive drug delivery devices, wound dressings, foot dressings, and fixation aids for human incontinence devices, medical devices (for example catheters, cannulas), ostomy bags and prosthetics (for example breasts and limbs).

### BACKGROUND OF THE INVENTION

Cross-linked conductive polymeric hydrogels have been used in medical devices, such as biomedical electrodes, to adhere the device to mammalian skin, to provide a secure conductive connection between the device and the skin for stimulation or sensing purposes. Most known compositions are based on polymeric matrices that are ionic in nature. Similar compositions are also known generally to be useful as wound dressings. The presence of the ionic groups imparts polyelectrolyte behaviour to the hydrogel. However, in certain applications, the known hydrogel compositions possessing polyelectrolyte character have been found to have significant disadvantages.

U.S. Pat. No. 3,929,741 (the disclosure of which is incorporated herein by reference) discloses anionic hydrogels based on 2-acrylamido-2-methylpropane sulphonic acid (AMPS) and its salts for use in contact lenses and wound dressings.

U.S. Pat. No. 4,391,278 (the disclosure of which is incorporated herein by reference) discloses anionic hydrogels based on 2-acrylamido-2-methylpropane sulphonic acid (AMPS) and its salts for use in biomedical electrodes.

U.S. Pat. No. 5,800,685 (the disclosure of which is incorporated herein by reference) discloses cationic hydrogels based on acrylic esters of quaternary chlorides or sulphates or acrylic amides of quaternary chlorides for use in biomedical electrodes. Copolymers of cationic and anionic monomers are mentioned generally, but not exemplified. The hydrogels are disclosed to be polyelectrolytes.

The gels disclosed in the above publications are considered to possess generally polyelectrolyte behaviour. Gels that are polyelectrolytes can potentially absorb significant quantities of aqueous solution and can then lose their as-made structural integrity and hence their usefulness. In mammalian skin contact applications the aqueous fluid may for example arise from sweat, exudates from wounds and bathing media.

WO-A-91/15250 (the disclosure of which is incorporated herein by reference) discloses amphoteric hydrogels formed from the copolymerisation of monomers possessing pendant strong acid groups, for example sulphonic acid, with monomers possessing pendant groups which are salts of strong basic groups, for example quaternary ammonium salts where at least one of the monomers is an N-substituted acrylamide. Many of the gels exemplified would be expected to exhibit polyampholytic characteristics as a consequence of global ionic balance. In the one example where the monomer with the pendant sulphonic acid is present as a salt, the resulting hydrogel is not ionically balanced and hence would be expected to exhibit polyelectrolyte behaviour.

U.S. Pat. No. 5,846,558 (the disclosure of which is incorporated herein by reference) discloses hydrogels based on polymers and copolymers of zwitterionic monomers. The anion and cation are carried on the same molecule in these monomers. No disclosure is made of any uptake of aqueous fluids.

It is an object of the present invention to provide in at least some embodiments ionic polymeric hydrogels possessing at least some resistance to the uptake of aqueous fluids possessing from zero to high ionic strength.

In such embodiments of the invention, the uptake of pure water and the uptake of ion-containing water (e.g. saline) can be balanced as desired, according to intended uses of the hydrogels.

It is a further object of the present invention to provide in at least some embodiments ionic hydrogels exhibiting relatively low absorption of certain types of aqueous fluids and also relatively high moisture vapour transmission rates.

It is a further object of the present invention to provide in at least some embodiments ionic polymeric hydrogel adhesives that can be used in diverse applications, for example medical devices including biomedical electrodes, ostomy, incontinence devices, skin contact devices for the delivery of medicaments, footcare and prosthetics.

It is a further object of the present invention to provide in at least some embodiments polymeric hydrogels with low aqueous fluid uptake in the form of films, more preferably a significantly reduced aqueous fluid uptake in comparison with known polymeric hydrogel films.

It is a further object of the present invention to provide in at least some embodiments polymeric hydrogels with significantly reduced aqueous fluid uptake in the form of foams and foam/film composites, in comparison with known polymeric hydrogels in the form of foams and foam/film composites.

### BRIEF DESCRIPTION OF THE INVENTION

According to the present invention, we provide a cross-linked polymeric hydrogel suitable for use in mammalian body tissue (e.g. skin) contacting applications, which comprises a cross-linked copolymer formed from a first monomer comprising one or more pendant anionic group and a second monomer comprising one or more pendant cationic group, the relative amounts of the said monomers in the copolymer being such that the anionic groups and cationic groups are present in essentially equimolar quantities.

The hydrogel is preferably a plasticised hydrogel, suitably containing an effective amount of one or more organic plasticiser. One or more polymeric or non-polymeric polyhydric alcohol, such as glycerol, are preferred as organic plasticisers. Other organic plasticisers can include, for example, polymeric alcohols, esters of polyhydric alcohols, esters of polyhydric alcohols and boric acid (e.g. a glycerol/boric acid ester).

The hydrogel suitably contains water as a further ingredient. Further relatively minor ingredients may also be present, as set forth in more detail below.

In one embodiment, the said anionic and cationic groups may be selected from groups which are salts of acid groups and groups which are salts of basic groups.

It is preferred that the copolymer is formed by the simultaneous crosslinking and copolymerising of the monomers, in suitable amounts whereby the molar ratio of anionic to cationic groups in the copolymer is substantially unity.

We have found that acceptable ionic mobility is present in the hydrogels according to the present invention, without the need for additional ions (e.g. ions from salts introduced into the polymerisation reaction mixture or the hydrogel) to impart this property. Additional ingredients may also be present in the hydrogel composition according to the present invention. Such additional ingredients may, for example, include one or more ionic and/or non-ionic compounds, such as medicaments (for example: antiseptics, antimicrobial agents, antibiotics, analgesics, anaesthetics), humectants (for example: glycerol, sorbitol, polyethylene glycol, methyl ether terminated polyethylene glycol), vitamins, adhesion enhancers (for example: vinyl acetate dioctylmaleate copolymers), pH buffers, citric acid, salicylic acid, surfactants and water soluble polymers (for example: polysaccharides and synthetic polymers).

The pendant groups in the first monomer are preferably the sodium, potassium, calcium, lithium and/or ammonium (individually or in any combination of one or more) salts of carboxylic acid, phosphoric acid and/or sulphonic acid. Sulphonic acid groups are most preferred. The pendant groups in the second monomer are preferably quaternary ammonium salts of halide (for example chloride), sulphate and/or hydroxide. Chloride and sulphate are most preferred.

The hydrogels of the present invention can suitably be made in aqueous solution by polymerisation of the first and second monomers, optionally with additional monomers. Any additional monomer may, for example, be a non-ionic monomer. The nature and proportions of any such additional monomers should be such that the polyampholytic characteristics of the final hydrogel, deriving from the substantially equimolar amounts of anionic and cationic monomers, are not substantially disrupted. Non-limiting examples of suitable additional monomers include hydroxyethyl acrylate and methacrylate, acryloyl morpholine, acrylic acid, vinyl pyrrolidone (N-vinyl pyrrolidone), polyethylene acrylates and methacrylates, acrylamide and N-substituted acrylamides and soya bean epoxy acrylate and any combination thereof. Further examples of suitable additional non-ionic monomers also include di-, tri-, and multi functional crosslinking agents, for example polyethylene glycol diacrylate (molecular weight between about 100 and 10,000) and methylene bisacrylamide, and mixtures thereof.

The pre-polymerisation (pregel) mixture may contain one or more surfactant if desired. For further details of suitable surfactants, please refer to the section headed “Surfactant” on pages 17 to 19 of our PCT patent application no. WO-A-00/46319, the contents of which are explicitly incorporated herein by reference as part of the disclosure of the present invention.

Simultaneously cross-linking and polymerising the monomers in aqueous solution by conventional free radical polymerisation utilising appropriate polymerisation catalysts can make the hydrogels of the present invention. Such free radical polymerisation may be initiated by any suitable initiation method, for example thermal, redox, ultra-violet light, gamma irradiation and electron beam initiation, which methods are known by those skilled in the art. Ultra-violet light initiated polymerisation is the preferred method. The polymerisation mixture preferably includes appropriate amounts of one or more initiator to assist the initiation process. For further details of suitable crosslinking agents and polymerisation ingredients and conditions see our PCT patent application no. WO-A-00/46319, particularly the section headed “Crosslinking and Polymerisation” from pages 8 to 11, the contents of which are explicitly incorporated herein by reference as part of the disclosure of the present invention.

The hydrogels of the present invention can be made by initially depositing the uncured pregel mixture, preferably as a layer, e.g. by casting onto a suitable member, such as a porous or non-porous film, net or non-woven material which are suitably made from synthetic materials, natural materials or mixtures of both. The deposited pregel may suitably be in the form of a continuous film, or as islands, or as a foam layer or body. The cured hydrogels can also be made to encapsulate porous or foraminous materials such as non-wovens and nets.

By suitable control of the ingredients of the hydrogels, and more particularly the relative amounts of the organic plasticiser and the monomers present, the relative uptake of pure water compared with saline exhibited by the hydrogels can be controlled. In this way, hydrogels having polyampholyte properties (preferential saline uptake) can, for example, be prepared for particular applications. Thus, for example, a polyampholytic hydrogel having a strong preference for saline in comparison with pure water can serve as an effective bioadhesive for a rain-, fresh-water-sport-, bath-, or shower-resistant wound or burn dressing (saline body exudates will generally challenge the hydrogel in relatively small amounts in comparison with the fresh water).

The invention therefore makes available the use of a hydrogel as previously defined as the bioadhesive portion of a medical, health or personal care product having a bioadhesive portion adapted for attachment of the product to mammalian body tissue such as skin. Such a product may be selected from: devices, sensing electrodes, stimulation electrodes, devices for iontophoretic delivery of active agents, passive drug delivery devices, wound dressings, foot dressings, and fixation aids for human incontinence devices, medical devices (for example catheters, cannulas), ostomy bags and prosthetics (for example breasts and limbs). The major part of the product is of generally conventional construction for that product. The bioadhesive portion may suitably be a film or sheet, but may take any convenient form provided that the bioadhesive effect is available. The bioadhesive portion is suitably protected prior to use, by means of a removable release sheet of conventional material (e.g. siliconised paper or plastic). Such a use and the products themselves constitute further aspects of the present invention.

### DETAILED DESCRIPTION OF THE INVENTION

As is set forth above, the present invention relates to conductive and adhesive hydrogels for use in a variety of applications involving contact with mammalian skin. These hydrogels possess a significantly reduced aqueous fluid uptake compared to those previously known in the art.

The anionic monomer is preferably 3-sulphopropyl acrylate (SPA) or a salt or analogue thereof, 2-acrylamido-2-methylpropane sulphonic acid (AMPS) or a salt or analogue thereof, or a mixture of both. The term “analogue” in this context refers particularly to substituted derivatives of SPA or 2-acrylamido-2-methylpropane sulphonic acid or salts thereof. The anionic monomer is preferably an alkali metal (e.g. sodium or potassium) salt of SPA or of 2-acrylamido-2-methylpropane sulphonic acid, e.g. sodium AMPS (or NaAMPS).

The cationic monomer is preferably either a quaternary ammonium salt derivative of acrylic acid or a quaternary ammonium salt derivative of an N-substituted acrylamide or combinations of both. Preferred examples include acryloyloxyethyltrimethyl ammonium chloride (e.g. DMAEQA-Q, Kohjin), acryloyloxyethyltrimethyl ammonium methyl sulphate (available, for example, from Aldrich), acrylamidopropyltrimethyl ammonium chloride (available, for example, from Kohjin).

Generally speaking, the weight ratio of the cationic monomer to the anionic monomer in the hydrogel may suitably lie within the range of about 0.85:1 to about 1.2:1.

It is particularly preferred that the hydrogel exhibits polyampholyte rather than polyelectrolyte behaviour. By polyelectrolyte behaviour we generally mean the fluid uptake property which is characterised by pure water being absorbed more readily than saline. By polyampholyte behaviour we generally mean the fluid uptake property which is characterised by saline being absorbed more readily than pure water.

We have found that polyampholyte behaviour in the ionically balanced hydrogels of the present invention generally requires a total cationic and anionic monomer content in the hydrogel at a level of greater than about 42% by weight of the hydrogel and/or at a weight ratio of organic plasticiser to total (cationic and anionic) monomer in the hydrogel at a level greater than about 1:3, for example greater than about 1:2 or greater than about 3:4.

The total amount of ionic monomer present in the hydrogel pre-polymerisation mix for making a film is about 0.2-60%, for example about 1-60%, preferably about 10-45%, and more preferably, about 20-45% by weight of the total composition, such that the molar ratio of anionic to cationic monomer is preferably from about 0.8 to about 1.2, preferably about 0.9 to about 1.1, more preferably about 0.95 to about 1.05 and more preferably about 1. The balance of the composition, preferably comprises water, preferably about 10 to about 80%, and more preferably about 15 to about 40%, a polyhydric alcohol 0 to about 50%, preferably about 10 to about 40%, where the polyhydric alcohol is preferably glycerol (available, for example, from Aldrich); a cross-linking agent about 0.04% to about 2%, preferably about 0.06 to about 0.3%, where the preferred crosslinking agent is polyethylene glycol diacrylate (available, for example, from Aldrich); a photoinitiator (e.g. Darocure 1173 or Irgacure 184 or combinations of both) preferably about 0.001% to about 0.1%; and additional additives, for example medicaments, adhesion promoters, 0% to about 10%.

The total amount of ionic monomer present in the hydrogel pre-polymerisation mik for making a foam is about 0.2-60%, for example about 1-60%, preferably about 10-45%, and more preferably about 20-45% by weight of the total composition, such that the molar ratio of anionic to cationic monomer is preferably from about 0.8 to about 1.2, preferably about 0.9 to about 1.1, more preferably about 0.95 to about 1.05 and more preferably about 1. The balance of the composition comprises water, preferably about 10 to about 80%, and more preferably about 15 to about 40%; a polyhydric alcohol 0 to about 50%, preferably about 10 to about 40%, where the polyhydric alcohol is preferably glycerol (available, for example, from Aldrich); a cross-linking agent about 0.04% to about 2%, preferably about 0.06 to about 0.3%, where the preferred crosslinldng agent is polyethylene glycol diacrylate (available, for example, from Aldrich); a photoinitiator (e.g. Darocure 1173 or Irgacure 184 or combinations of both) preferably about 0.001% to about 0.1%; surfactant about 0.001% to about 10% where the surfactant is preferably non-ionic, for example a Pluronic from Ciba Geigy (P65, L64); and additional additives, for example medicaments, adhesion promoters, 0% to about 10%.

In one embodiment, the hydrogel compositions according to the present invention consist essentially of the defined cross-linked copolymer according to the general definition of the invention stated above in the Brief Description of the Invention, together with one or more of water, and optionally one or more of a surfactant and a humectant or plasticiser (e.g. a polyhydric alcohol), with less than about 10%, more typically less than about 8%, more preferably less than about 5%, of other ingredients such as one or more of medicaments and adhesion promoters. The proportions of the ingredients are preferably as stated above.

All percentages of ingredients are given by weight.

The process for mixing the ingredients of the hydrogel and curing (polymerising) the pregel mixture will be well understood by those of ordinary skill in the art. Please refer to our PCT patent application no. WO-A-00/46319, particularly the section headed “Polymerisation Conditions” on pages 21 to 22, for further details. This section of the prior art reference is explicitly incorporated herein by reference as part of the disclosure of the present invention.

From the assembly of the pre-polymerisation mix, a continuous film is preferably made by coating the mix onto a substrate, preferably siliconised for easy release, such as polyester, polyethylene, polypropylene, polyurethane, paper or a net, foam or a non woven material made from natural and/or synthetic materials, and passed under a UV light for curing. After curing a siliconised cover is placed on top of the exposed surface of the hydrogel. The thickness of the hydrogel film can be from about 0.05 mm to about 3 mm.

A foamed hydrogel of the present invention can suitably be made by mechanically agitating the premix and then coating on to web as for the film. The foam so formed can be porous throughout its thickness, or can be coated such that a composite structure of film supporting a foam can be made. The thickness of the foam or film foam structure can suitably be from about 0.1 mm to about 3 mm.

The hydrogels according to the present invention preferably show a total fluid weight uptake at a level up to about 2000% in 24 hours, more preferably up to about 1000%. This is the weight of fluid taken up in the stated time on contact with fluid, expressed as a percentage of the weight of initial hydrogel.

As stated above, the hydrogels of the present invention can be tailored to exhibit preferential uptake of pure water in comparison to saline, or vice versa. The pure water to saline weight uptake ratio over 24 hours can typically lie within the range of about 2:1 to about 1:2 in hydrogels of the present invention.

### EXAMPLES OF THE INVENTION

The following Examples are included purely for illustration of the present invention, without limitation.

### Example 1

40.84 g of a 58% aqueous solution of NaAMPS (Lubrizol) were mixed with 25 g of a 79% aqueous solution of DMAEA-Q (Kohjin) and 34.16 g of glycerol for 30 minutes. To this mixture 0.19 g of a Darocure 1173 photoinitiator (4 parts) and polyethylene glycol diacrylate (IRR 280, UCB) (20 parts) solution was added and stirred for 30 minutes. The mixture was then coated on to a siliconised polyester backing and passed under a UV lamp. The mixture cured rapidly to produce a gel with good tack and adhesion properties. The gel had low saline uptake.

### Example 2

20.42 g of a 58% aqueous solution of NaAMPS (Lubrizol) and 20.44 g of a 58% aqueous solution of SPA (potassium salt) (Raschig) were mixed with 25 g of a 79% aqueous solution of DMAEA-Q (Kohjin) and 34.16 g of glycerol for 30 minutes. To this mixture 0.19 g of a Darocure 1173 photoinitiator (4 parts) and polyethylene glycol diacrylate (IRR 280, UCB) (20 parts) solution was added and stirred for 30 minutes. The mixture was then coated on to a siliconised polyester backing and passed under a UV lamp. The mixture cured rapidly to produce a gel with good tack and adhesion properties. The gel had low saline uptake.

### Example 3

20.42 g of a 58% aqueous solution of NaAMPS (Lubrizol) were mixed with 12.5 g of a 79% aqueous solution of DMAEA-Q (Kohjin) and 67.08 of glycerol for 30 minutes. To this mixture 0.25 g of a Darocure 1173 photoinitiator (4 parts) and polyethylene glycol diacrylate (IRR 280, UCB) (20 parts) solution was added and stirred for 30 minutes. The mixture was then coated on to a siliconised polyester backing and passed under a UV lamp. The mixture cured rapidly to produce a gel with good tack and adhesion properties. The gel had low saline uptake.

### Example 4

40.84 g of a 58% aqueous solution of SPA (potasium salt) (Raschig) were mixed with 25 g of a 79% aqueous solution of DMAEA-Q (Kohjin) and 34.16 g of glycerol for 30 minutes. To this mixture 0.19 g of a Darocure 1173 photoinitiator (4 parts) and polyethylene glycol diacrylate (IRR 280, UCB) (20 parts) solution was added and stirred for 30 minutes. The mixture was then coated on to a siliconised polyester backing and passed under a UV lamp. The mixture cured rapidly to produce a gel with good tack and adhesion properties. The gel had low saline uptake.

### Example 5

40.84 of a 58% aqueous solution of NaAMPS (Lubrizol) were mixed with 25 g of a 79% aqueous solution of DMAEA-Q (Kohjin) and 34.16 g of glycerol for 30 minutes and 3 g of Pluronic P65 (Ciba Geigy). To this mixture 0.19 g of a Darocure 1173 photoinitiator (4 parts) and polyethylene glycol diacrylate (IRR 280, UCB) (20 parts) solution was added and stirred for 30 minutes. The mixture was mechanically agitated to produce foamed liquid and then coated on to a siliconised polyester backing and passed under a UV lamp. The mixture cured rapidly to produce a gel with good tack and adhesion properties. The gel had low saline uptake compared to gel made using the same method but replacing the DMAEA-Q with NaAMPS.

### Example 6

Samples of hydrogels according to the invention, containing varying amounts of glycerol, were tested for their relative uptakes of fresh water and 0.9% saline in parallel tests. Water uptake is measured in weight %, i.e. weight of water/saline in grams absorbed in 24 hours per 100 grams of initial hydrogel.

The results are shown in Table 1;

TABLE 1

| | | | Water | 0.9% NaCl |
|---|---|---|---|---|
| % DMAEA-Q | % NaAMPS | % Glycerol | Uptake (%) | Uptake (%) |
| 18 | 21.11 | 0 | 712 | 536 |
| 18 | 21.11 | 10 | 512 | 411 |
| 18 | 21.11 | 20 | 419.5 | 462 |
| 18 | 21.11 | 30 | 201 | 502 |

DMAEA-Q is N,N-dimethylaminoethylacrylate, methyl chloride quaternary. The level of PI/XL in the gels was 0.1% 4/20.

Two trends emerge. Firstly as the glycerol content increases the water uptake decreases, the gel seems to behave more like a non-ionic gel with very low swelling in water. The second trend is that at high glycerol content the saline uptake, while quite low, is actually higher than the water uptake. At low glycerol content saline uptake is lower than water uptake. However, if the water and salt uptake experiments are undertaken over a longer period of time the water uptake becomes greater than the salt uptake for gels produced with glycerol.

The foregoing broadly defines the present invention without limitation. Variations and modifications as will be readily apparent to those of ordinary skill in this art are intended to be included within the scope of this application and subsequent patents.

1. A cross-linked polymeric hydrogel suitable for use in mammalian body tissue contacting applications, which comprises a cross-linked copolymer formed from a first monomer comprising one or more pendant anionic group and a second monomer comprising one or more pendant cationic group, the relative amounts of the said monomers in the copolymer being such that the anionic groups and cationic groups are present in essentially equimolar quantities.

2. A hydrogel according to claim 1, further containing an effective amount of one or more organic plasticizer.

3. A hydrogel according to claim 2, wherein the organic plasticizer comprises glycerol.

4. A hydrogel according to claim 1, further containing water.

5. A hydrogel according to claim 1, further containing one or more other ingredients in minor amounts wherein the said one or more other ingredients is selected from the group consisting of: one or more ionic and/or non-ionic compounds, humectants, vitamins, adhesion enhancers, pH buffers, citric acid, salicylic acid, surfactants and water soluble polymers.

6. (canceled)

7. A hydrogel according to claim 1, wherein the said anionic and cationic groups are selected from the group consisting of groups which are salts of acid groups and groups which are salts of basic groups.

8. A hydrogel according to claim 1, wherein the copolymer is formed by the simultaneous crosslinking and copolymerising of the monomers, in suitable amounts whereby the molar ratio of anionic to cationic groups in the copolymer is substantially unity.

9. A hydrogel according to claim 1, wherein the pendant groups in the first monomer are selected from the sodium, potassium, calcium, lithium and/or ammonium salts of carboxylic acid, phosphoric acid and/or sulphonic acid.

10. A hydrogel according to claim 9, wherein the first monomer is 3-sulphopropyl acrylate (SPA) or a salt or analogue thereof, 2-acrylamido-2-methylpropane sulphonic acid (AMPS) or a salt or analogue thereof, or a mixture of both.

11. A hydrogel according to claim 10, wherein the first monomer is an alkali metal salt of SPA or of 2-acrylamido-2-methylpropane sulphonic acid.

12. A hydrogel according to claim 1, wherein the pendant groups in the second monomer are quaternary ammonium salts of halide, sulphate and/or hydroxide.

13. A hydrogel according to claim 12, wherein the second monomer is a quaternary ammonium salt derivative of acrylic acid or a quaternary ammonium salt derivative of an N-substituted acrylamide or combinations of both.

14. A hydrogel according to claim 13, wherein the second monomer is acryloyloxyethyltrimethyl ammonium chloride, acryloyl-oxyethyltrimethyl ammonium methyl sulphate, or acrylamidopropyltrimethyl ammonium chloride.

15. A hydrogel according to claim 1, wherein the weight ratio of the cationic monomer to the anionic monomer in the hydrogel lies within the range of about 0.85:1 to about 1.2:1.

16. A hydrogel according to claim 1, wherein the hydrogel exhibits polyampholyte rather than polyelectrolyte behaviour.

17. A hydrogel according to claim 1, wherein the total cationic and anionic monomer content in the hydrogel is greater than about 42% by weight of the hydrogel.

18. A hydrogel according to claim 2, wherein the weight ratio of organic plasticiser to total cationic and anionic monomer in the hydrogel is greater than about 1:3.

19. A hydrogel according to claim 1 having a total fluid weight uptake of up to about 2000% in 24 hours.

20. A hydrogel according to claim 1, having a pure water to saline weight uptake ratio over 24 hours within the range of about 2:1 to about 1:2.

21. A method of use of a hydrogel according to claim 1, as the bioadhesive portion of a medical, health or personal care product having a bioadhesive portion adapted for attachment of the product to mammalian body tissue, optionally skin.

22. The method according to claim 21, wherein the product is selected from the group consisting of: devices, sensing electrodes, stimulation electrodes, devices for iontophoretic delivery of active agents, passive drug delivery devices, wound dressings, foot dressings, and fixation aids for human incontinence devices, medical devices (optionally catheters, cannulas), ostomy bags and prosthetics (optionally breasts and limbs).

23. A medical, health or personal care product, comprising a portion of generally conventional construction for the said product and a bioadhesive portion adapted for attachment of the product to mammalian body tissue, optionally skin, wherein the said bioadhesive portion comprises a hydrogel as defined in claim 1.

24. A medical, health or personal care product according to claim 23, wherein the product is selected from the group consisting of: devices, sensing electrodes, stimulation electrodes, devices for iontophoretic delivery of active agents, passive drug delivery devices, wound dressings, foot dressings, and fixation aids for human incontinence devices, medical devices (optionally catheters, cannulas), ostomy bags and prosthetics (optionally breasts and limbs).

25. The hydrogel of claim 5 wherein said one or more other ingredients is selected from the group consisting of medicaments including antiseptics, antimicrobial agents, antibiotics, analgesics and anesthetics; glycerol; sorbitol; polyethylene glycol; methyl ether terminated polyethylene glycol; vinyl acetate dioctylmaleate copolymers; polysaccharides and synthetic polymers.

26. The hydrogel of claim 2 having a total fluid weight uptake of up to about 1,000%.
